(19) United States
(12) Patent Application Publication
(10) Pub. No.: US 2026/0022081 A1
(43) Pub. Date: Jan. 22, 2026

(54) A PROCESS TO PREPARE KEROSENE

(71) Applicant: SHELL USA, INC., Houston, TX (US)

(72) Inventors: Coen Bernhard HODES, Amsterdam (NL); Leon Bernardus Johannes VAN DEN ENK, Amsterdam (NL); Tyrone James MCKNIGHT, Amsterdam (NL); Andries Hendrik JANSSEN, Amsterdam (NL); Edward Julius CREYGHTON, Amsterdam (NL)

(21) Appl. No.: 18/869,261

(22) PCT Filed: Jun. 21, 2023
(86) PCT No.: PCT/EP2023/066792

§ 371 Date: Nov. 25, 2024

(30) Foreign Application Priority Data
Jun. 22, 2022 (EP) 22180578.1

(51) Int. Cl. C07C 1/12

(52) U.S. Cl. CPC C07C 1/12

(57) ABSTRACT

The present invention provides a process to prepare kerosene, the process at least comprising the steps of: (a) providing a syngas stream comprising hydrogen (H₂) and carbon monoxide (CO); (b) subjecting the syngas stream provided in step (a) to a Fischer-Tropsch reaction thereby obtaining a Fischer-Tropsch product comprising at least 50 wt. % of compounds boiling above 370° C.; (c) separating the Fischer-Tropsch product into at least a C1-C4 fraction, H₂O and a C5+ fraction; (d) subjecting the C5+ fraction as separated in step (c) to hydroprocessing thereby obtaining a mixture comprising at least a kerosene fraction and a heavier fraction; (e) separating the mixture as obtained in step (d) thereby at least obtaining the kerosene fraction and the heavier fraction; (f) recycling at least a part of the heavier fraction as obtained in step (e) to the hydroprocessing of step (d).

### FIELD OF THE INVENTION

The present invention relates to a process to prepare kerosene, in particular from a Fischer-Tropsch derived product obtained from syngas.

### BACKGROUND OF THE INVENTION

It is known in the art to prepare one or more middle distillate fractions such as for example kerosene or gas oil and a base oil precursor or a base oil from a Fischer-Tropsch derived feedstock.

As an example, WO 2015/063213 A1 discloses a process wherein one or more middle distillate fractions and a first residual fraction are obtained by using two different catalysts in series in the hydroprocessing of a Fischer-Tropsch derived feedstock, wherein both catalysts have hydrocracking and hydroisomerising activity and the second catalyst is more active in hydroisomerisation and less active in hydrocracking compared to the first catalyst. Stacked bed in WO 2015/063213 A1 allows for the preparation of lubricating base oils and one or more middle distillate fractions with improved cold flow properties.

As a further example, WO 2021/255145 A1 discloses a process to prepare middle distillates and base oils from a Fischer-Tropsch product. Experimental B and Example 4 of WO 2021/255145 A1 show that middle distillates can be produced in addition to base oils in a line-up with an atmospheric distillation operating at an effective cut point of about 370° C. and recycling material boiling above about 540° C. obtained from a vacuum distillation step.

Example 4 of WO 2021/255145 A1 does not provide any indication of (high) yields of kerosene as only the yield of a 175-370° C. middle distillate fraction is shown (in Table 4). The 370° C. end point of this middle distillate fraction is too high for kerosene, i.e. the 175-370° C. middle distillate fraction will comprise gasoil and not only kerosene: the total yield of middle distillates (kerosene and gasoil) as indicated in Table 4 on page 21 of WO 2021/255145 A1 is 40.4 wt. % As another example, WO 2006/053894 A1 discloses a process to optimize the yield of gas oils. As indicated on page 11, lines 26-30 of WO 2006/053894 A1, the kerosene fraction preferably boils for more than 80 wt. % between 175 and 250° C.

US 2004/067843 A1 discloses oxygenate treatment of dewaxing catalysts for greater yield of dewaxed product.

No kerosene or naphtha yields are reported.

Recently, increased focus has been on producing kerosene from renewable feedstocks.

This as, as explained in for example WO 2020/154810, jet fuel is the least likely of the transportation fuels to be replaced by non-hydrocarbon based fuels, such as electricity. To this end, WO 2020/154810 A1 proposes a process for producing synthetic jet fuel comprising: converting feedstock to synthesis gas; converting the synthesis gas into a mixture comprising liquid hydrocarbons; refining the mixture comprising liquid hydrocarbons to isolate a kerosene product; and hydrotreating the kerosene product to form synthetic jet fuel. From paragraph [00142] of WO 2020/154810 A1 it is clear that at least four product fractions are obtained: a) an aqueous product; b) a naphtha and gas product; c) a kerosene product; and d) a gas oil and heavier product.

In the examples of WO 2020/154810 A1, several catalysts are mentioned: Examples 1 and 2 mention a non-sulfided H-ZSM-5 oligomerization catalyst and a reduced, non-sulfided Ni/Al₂O₃hydrotreating catalyst; Example 3 mentions a Pt/SiO₂—Al₂O₃ hydrocracking catalyst. Oligomerization and hydrocracking take place at about 1.5 MPa to 3 MPa

The final output of the process of WO 2020/154810 A1 (cf. paragraph [00174]) is a jet fuel having a high boiling point (e.g. between 140 to 260° C.) and a low freezing point (e.g. below −60° C.). Such a low freezing point is not optimal, as it is much lower than required according to Jet A-1 fuel standards (−47° C.). As also acknowledged in paragraph [00181], jet fuel yields as obtained according to the process of WO 2020/154810 A1 (focusing on a jet fuel product with a high boiling point (between 140 to 260° C.) and a low freezing point (below −60° C.)) can be optimized.

In this respect it is noted that paragraph [00187] of WO 2020/154810 A1 mentions, whilst discussing hydrocracking of a wax that: “Of interest to manufacturing of synthetic jet fuel, is the selectivity ratio of kerosene to naphtha. At the operating conditions employed herein, the mass ratio of hydrocarbons in the 140-260° C. boiling range to hydrocarbons with boiling point <140° C., was 1:1.”. This mass ratio of 1:1 means that 50 wt. % of the hydrocracked products with a boiling range below 260° C. is gas and naphtha. This means that higher kerosene yields are possible.

Hence, a problem of the process of WO 2020/154810 A1 is that the yield of kerosene is limited.

A further problem of the process according to WO 2020/154810 A1 is that an increase in the yield of kerosene is being achieved through oligomerization and hydrotreating steps, thereby requiring several different process units with many pieces of processing equipment.

US 2014/005450 A1 discloses the use of n-paraffin adsorption to increase selectivity and yield of synthetic distillate fuel. Paragraph [0015] mentions a cut point of typically between 254 and 266° C.

### SUMMARY OF THE INVENTION

It is an object of the present invention to solve or minimize at least one of the above or other problems.

It is a further object of the present invention to provide a process to produce premium quality kerosene, meeting the Jet A-1 specifications (as defined by ASTM D7566-20).

Another object of the present invention is to provide an alternative process to produce premium kerosene in high yields (e.g. above 70%), without the need for (or use of) additional catalytic dewaxing or additional oligomerization.

One of the above or other objects may be achieved according to the present invention by providing a process to prepare kerosene, the process at least comprising the steps of:
(a) providing a syngas stream comprising hydrogen (H₂) and carbon monoxide (CO);
(b) subjecting the syngas stream provided in step (a) to a Fischer-Tropsch reaction thereby obtaining a Fischer-Tropsch product comprising at least 50 wt. % of compounds boiling above 370° C.;
(c) separating the Fischer-Tropsch product into at least a C1-C4 fraction, H₂O and a C5+ fraction; (d) subjecting the C5+ fraction as separated in step (c) to hydroprocessing thereby obtaining a mixture comprising at least a kerosene fraction and a heavier fraction;
(e) separating the mixture as obtained in step (d) thereby at least obtaining the kerosene fraction and the heavier fraction, wherein the kerosene fraction has a Final Boiling Point of at most 302° C., preferably at most 300° C. as determined by ASTM D86-18 and a flashpoint of at least 38° C. as determined by IP170,
wherein in the separation of step (e) the effective cut point, as determined by ASTM D2892-15 X2, between the kerosene fraction and the heavier fraction is at least 315° C. and at most 330° C., and
wherein in step (e) a kerosene yield of above 70 wt. % is obtained, based on the weight of the C5+ fraction subjected to hydroprocessing in step (d);
(f) recycling at least a part of the heavier fraction as obtained in step (e) to the hydroprocessing of step (d);
wherein step d) is carried out by contacting the C5+ fraction with a first catalyst, wherein the first catalyst comprises a molecular sieve with a pore size between 5 and 7 angstrom and a SiO₂/AlO₃ ratio of at least 25 and a group VIII metal.

It has now surprisingly been found according to the present invention that by using a catalyst comprising a molecular sieve with a pore size between 5 and 7 angstrom and a SiO₂/AlO₃ ratio of at least 25 and a group VIII metal in the hydroconversion step of a Fischer-Tropsch derived feedstock, and by recycling a relatively wide-cut fraction (i.e. the fraction heavier than kerosene), a high kerosene yield (>70 wt. %) can be obtained.

A further advantage of the process according to the present invention is that it results in a kerosene fraction with a relatively low aromatics content (meeting the maximum 0.5 wt. % aromatics as specified in ASTM D7566-20, Annex A1).

Furthermore, with the process according to the present invention the kerosene can be obtained in a single hydrocracking step, without a separate catalytic dewaxing or oligomerization step (i.e. less CAPEX, no additional yield loss).

An important advantage of the present invention is that premium kerosene is obtained that qualifies for jet fuel from a freezing point perspective, i.e. having a freezing point below −40° C. (Jet A), preferably below −47° C. (Jet A1).

### DETAILED DESCRIPTION OF THE INVENTION

In step (a) of the process according to the present invention, a syngas stream is provided that comprises hydrogen (H₂) and carbon monoxide (CO).

As the person skilled in the art is familiar with how to provide a syngas stream, this is not discussed here in detail. It goes without saying that various (hydro)carbonaceous feedstocks can be used to provide the syngas stream. Suitable feedstock include natural gas, crude oil, heavy oil fractions, coal, biomass, waste and lignite. Preferably, the feedstocks as used in the present invention have a renewable origin such as biomass, waste, lignite and captured CO₂ (from industrial, biological sources or by means of Direct Air Capture). The H₂ in the syngas stream may have been produced using renewable sources such as wind and solar energy.

In step (b) of the process according to the present invention, the syngas stream provided in step (a) is subjected to a Fischer-Tropsch reaction thereby obtaining a Fischer-Tropsch product comprising at least 50 wt. % of compounds boiling above 370° C.

Again, as the person skilled in the art is familiar with Fischer-Tropsch reactions and Fischer-Tropsch products, this is not discussed here in detail.

By the term ‘Fischer-Tropsch product’ is meant a synthesis product of a Fischer-Tropsch reaction. A Fischer-Tropsch product derived from natural gas may also be referred to a GTL (Gas-to-Liquids) product. Fischer-Tropsch products derived from other feedstocks may also be referred to as XTL (Anything-to-Liquids) product. The preparation of a Fischer-Tropsch product by means of a Fischer-Tropsch reaction has been described in e.g. WO 2003/070857.

In step (c) of the process according to the present invention, the Fischer-Tropsch product is separated into at least a C1-C4 fraction, H₂O and a C5+ fraction.

Separation of a Fischer-Tropsch product is known in the art. Usually, according to the prior art, the Fischer-Tropsch product (which suitably comprises a C1 to C300 fraction) is separated into a water stream (which may contain oxygenated hydrocarbons), a gaseous stream comprising unconverted synthesis gas, carbon dioxide, inert gasses and several hydrocarbon fractions. Usually, lighter fractions of the Fischer-Tropsch product, which suitably comprise a C3 to C9 fraction are separated from the Fischer-Tropsch product by distillation thereby obtaining a Fischer-Tropsch product stream, which suitably comprises C10 to C300 fraction.

However, according to the present invention the whole C5+ fraction is separated and processed further.

The (‘wide-cut’) C5+ fraction comprises hydrocarbons containing 5 carbon atoms (i.e. C5) and heavier hydrocarbons. Preferably, the C5+ fraction comprises at least 1.0 wt. % of C5-C9, preferably at least 3.0 wt. % C5-C9.

The Fischer-Tropsch product used in the present invention is typically a paraffinic feedstock that comprises at least 50 wt. % of compounds boiling above 370° C. and which has a paraffin content of at least 60 wt. %, an aromatics content of below 1 wt. %, a naphthenic content of below 2 wt. %, a nitrogen content of below 0.1 wt. %, and a sulphur content of below 0.1 wt. %.

Preferably, at least a part of the C1-C4 fraction obtained in step (c) is converted into syngas and combined with the syngas stream provided in step (a).

This may contribute to an increased yield in the kerosene fraction.

In step (d) of the process according to the present invention, the C5+ fraction as separated in step (c)—and the heavier fraction as recycled in step (f) as a combined stream—is subjected to hydroprocessing thereby obtaining a mixture comprising at least a kerosene fraction and a heavier fraction.

As the person skilled in the art is familiar with hydroprocessing, this is not discussed here in detail. Hydroprocessing in step (d) may take place in a heavy paraffin conversion unit. In this unit, in the presence of the catalyst both hydrocracking and hydroisomerization takes place. The product stream is contacted in the presence of hydrogen, at a typical pressure in the range of 20 to 100 bara and at a temperature between 25° and 400° C. Preferably, the hydroprocessing in step (d) takes place at a pressure of above 20 bara, preferably above 30 bara, more preferably above 40 bara. Generally, the hydroprocessing in step (d) takes place at a pressure of below 100 bara, preferably below 70 bara. Preferably, the hydroprocessing in step (d) takes place at a pressure in the range of from above 30 to 70 bara and at a temperature between 30° and 400° C.

Hydrocracking/hydroisomerization processes are known in the art and therefore not discussed here in detail.

Hydrocracking/hydroisomerization and the effect of hydrocracking/hydroisomerization conditions on the amount of isomerised product are for example described in Chapter 6 of “Hydrocracking Science and Technology”, Julius Scherzer; A. J. Cruia, Marcel Dekker, Inc, New York, 1996, ISBN 0-8247-9760-4.

According to the present invention, step d) is carried out by contacting the C5+ fraction—and the heavier fraction as recycled in step (f) as a combined stream—with a first catalyst, wherein the first catalyst comprises a molecular sieve with a pore size between 5 and 7 angstrom and a SiO₂/AlO₃ ratio of at least 25 (preferably from 50 to 180) and a group VIII metal.

Suitably, the first catalyst used in step (d) of the process according to the present invention comprises a molecular sieve with a pore size between 5 and 6.6 angstrom.

Preferably, the first catalyst is a heterogeneous catalyst comprising molecular sieves, more suitably 10- or 12-membered ring molecular sieves with pore sizes between 5 and 6.6 angstrom, preferably monodimensional 10- or 12-membered ring molecular sieves with pore sizes between 5 and 6.6 angstrom, more preferably monodimensional 10- or 12-membered ring molecular sieves with pore sizes between 5 and 6.2 angstrom in combination with a metal having a hydrogenation function, such as the Group VIII metals. The indicated pore sizes relate to the largest diameter of the pores as described in the 6^{th} revised edition of the Atlas of Zeolite Framework Types published in 2007 on behalf of the Structure Commission of the International Zeolite Association.

Preferably, the first catalysts comprises a molecular sieve and a Group VIII metal, wherein the molecular sieve is selected from a group consisting of a MTW, MTT, TON type molecular sieve, ZSM-48 and EU-2.

In the present invention, the reference to ZSM-48 and EU-2 is used to indicate that all zeolites can be used that belong to the ZSM-48 family of disordered structures also referred to as the *MRE family and described in the Catalogue of Disorder in Zeolite Frameworks published in 2000 on behalf of the Structure Commission of the International Zeolite Association. Even if EU-2 would be considered to be different from ZSM-48, both ZSM-48 and EU-2 can be used in the present invention. Zeolites ZBM-30 and EU-11 resemble ZSM-48 closely and also are considered to be members of the zeolites whose structure belongs to the ZSM-48 family. In the present application, any reference to ZSM-48 zeolite also is a reference to ZBM-30 and EU-11 zeolite.

Besides ZSM-48 and/or EU-2 zeolite, further zeolites can be present in the catalyst composition especially if it is desired to modify its catalytic properties. It has been found that it can be advantageous to have present zeolite ZSM-12 which zeolite has been defined in the Database of Zeolite Structures published in 2007/2008 on behalf of the Structure Commission of the International Zeolite Association.

Suitable Group VIII metals are in particular nickel, cobalt, platinum and palladium. Preferably, the Group VIII metal is platinum or palladium.

Preferably, the first catalyst comprises a molecular sieve, and platinum or palladium as Group VIII metal, wherein the molecular sieve is a MTW, MTT, TON type molecular sieve or ZSM-48 or EU-2.

Suitably, the first catalyst used in step (d) is a dewaxing catalyst. The dewaxing catalyst suitably also comprises a binder. The binder can be non-acidic.

Examples of suitable binders are clay, silica, titania, zirconia, alumina, mixtures and combinations of the above and other binders known to one skilled in the art.

Preferably the catalyst comprises an alumina, silica or a titania binder.

Preparation of the dewaxing catalysts is for example described in WO 2015/063213 A1. If desired, a dealumination treatment of zeolites (if used) as described in WO 00/29511 A1 (in particular as described on page 10, line 10-page 11, line 31) can be considered.

According to an especially preferred embodiment according to the present invention, in step (d) the C5+ fraction—and the heavier fraction as recycled in step (f) as a combined stream—is contacted with a second catalyst before contacting with the first catalyst, wherein the second catalyst comprises a Group VIII noble metal supported on an amorphous acidic carrier.

Reference herein to an amorphous carrier is to a carrier not comprising a zeolitic or otherwise crystalline material. Preferred amorphous acidic carriers comprise refractory metal oxide carriers, more preferably silica, alumina, silica-alumina, zirconia, titania and mixtures thereof, even more preferably silica, alumina and silica-alumina.

A particularly preferred second catalyst comprises platinum supported on a silica-alumina carrier.

The second catalyst preferably comprises a Group VIII noble metal as hydrogenation/dehydrogenation functionality. The Group VIII noble metal preferably is palladium, platinum or a combination thereof, more preferably platinum. The second catalyst may comprise the Group VIII noble metal in an amount of from 0.005 to 5 parts by weight, preferably from 0.02 to 2 parts by weight, per 100 parts by weight of carrier material. A particularly preferred second catalyst comprises platinum in an amount in the range of from 0.05 to 2 parts by weight, more preferably from 0.1 to 1 parts by weight, per 100 parts by weight of carrier material. The second catalyst may also comprise a binder to enhance the strength of the catalyst. The binder can be non-acidic.

Examples are clays and other binders known to one skilled in the art. Examples of catalysts that may suitably be used as second catalyst are described in WO-A-0014179, EP-A-532118, EP-A-666894, EP-A-776959, and WO2009/080681.

According to another preferred embodiment according to the present invention, in step (d) the C5+ fraction—and the heavier fraction as recycled in step (f) as a combined stream—is contacted with a second catalyst after contacting with the first catalyst, wherein the second catalyst comprises a Group VIII noble metal supported on an amorphous acidic carrier.

Generally it is preferred, in case both the first and the second catalysts are present, that the amount of catalyst as used in step d) comprises 10-90 vol. % of the first catalyst and 90-10 vol. % of the second catalyst.

In step (e) of the process according to the present invention the mixture as obtained in step (d) is separated thereby at least obtaining the kerosene fraction and the heavier fraction, wherein the kerosene fraction has a Final Boiling Point of at most 302° C., preferably at most 300° C. as determined by ASTM D86-18 and a flashpoint of at least 38° C. as determined by IP170.

Please note in this respect that ASTM 7566-20 Annex A1 specifies other methods than IP170 as well for determining flashpoint, which other methods (e.g. ASTM D56) result in similar values.

The separation in step (e) is typically done by means of atmospheric distillation.

Preferably, the kerosene fraction obtained in step (e) has a freezing point of at most −40° C. as determined by ASTM D5972, preferably at most −47° C. In this way, the kerosene fraction as obtained in step (e) according to the process of the present invention has cold flow properties which properties make the kerosene fraction suitable as a jet-A or even jet-A1 blending component.

Please note in this respect that ASTM 7566-20 Annex A1 specifies also other methods than ASTM D5972 for determining the freezing point, which other methods (e.g. ASTM D2386) result in similar values.

Furthermore, it is preferred that the kerosene fraction obtained in step (e) has an amount of C16+ of at least 5 wt. %, preferably at least 10 wt. %, more preferably at least 15 wt. %, even more preferably at least 20 wt. %.

According to the present invention, in the separation of step (e) the effective cut point, as determined by ASTM D2892-15 X2, between the kerosene fraction and the heavier fraction is at least 315° C. and at most 330° C., preferably at most 325° C. Preferably, the effective cut point, again as determined by ASTM D2892-15 X2, between the kerosene fraction and the heavier fraction is at least 316° C., more preferably at least 317° C., even more preferably at least 318° C., yet even more preferably at least 319° C. and most preferably at least 320° C. In this respect it is noted that this effective cut point as determined by ASTM D2892-15 X2 is measured with a different method than the above-mentioned FBP as determined by ASTM D86-16 (and hence may lead to a different value).

The person skilled in the art will readily understand that in step (e)—in addition to the kerosene fraction and the heavier fraction—further fractions may be obtained from the mixture as obtained in step (d). Examples are a second C1-C4 fraction and a naphtha fraction.

According to an especially preferred embodiment of the present invention in step (e) also a second C1-C4 fraction and a naphtha fraction are obtained, wherein at least a part of the second C1-C4 fraction and/or the naphtha fraction are converted into syngas and combined with the syngas stream provided in step (a).

In the context of the present invention, the naphta fraction comprises at least 50 wt. % C5-C7, preferably at least 75 wt. % C5-C7.

According to the present invention, in step (e) a kerosene yield of above 70 wt. % is obtained, based on the weight of the C5+ fraction subjected to hydroprocessing in step (d).

In step (f) of the process according to the present invention at least a part of the heavier fraction as obtained in step (e) is recycled to the hydroprocessing of step (d). Preferably the majority (at least 50 wt. %), more than 90 wt. % or even all of the heavier fraction as obtained in step (e) is recycled to the hydroprocessing of step (d).

For the sake of clarity it is noted that this heavier fraction is combined with the C5+ fraction and subjected to hydroprocessing as a combined stream.

An important aspect of the process according to the present invention is that in step (e) a ‘wider cut’ is separated from the kerosene fraction (as the ‘heavier fraction’) and that this wider cut is recycled to the hydroprocessing step in step (d). In this respect it is noted that recycling of fractions obtained in a separation step has been proposed before, but such recycling would then be limited to ‘smaller cuts’ (such as the proposed recycling of the second residual fraction in WO 2021/255145 A1, the second residual fraction being a residual base oil).

Hereinafter the invention will be further illustrated by the following non-limiting examples.

### EXAMPLES

### Example 1 (Invention)

A Fischer-Tropsch product was obtained by separating the effluent from a Fischer-Tropsch process, in a series of hot- and cold-, high-pressure and low-pressure separators, in an off-gas stream comprising C1-C4 hydrocarbons, an aqueous stream containing dissolved oxygenated hydrocarbons, a light wax stream and a heavy wax stream.

The light wax stream and heavy wax stream were combined to form a C5+ fraction to be used in the experiment. The C5+ fraction contained 6.8 wt. % boiling below 150° C. and 58.9 wt. % boiling above 370° C. as determined by ASTM D-7169 and ASTM D-2887.

The whole C5+ fraction (i.e. a relatively ‘wide cut’) was continuously fed to a hydrocracking step operated at a pressure of 60 barg and a temperature of 341° C.

In the hydrocracking step the C5+ feed was contacted in a reactor with a silica-bound, ammonium hexafluorosilicate-treated Pt/ZSM-12 catalyst (a ‘first catalyst’ as meant according to the present invention) with a largest pore diameter of 6.0 angstrom and a SAR of 90. The weight hourly space velocity (based on the total volume of catalyst in the reactor) was 1.0 kg fresh feed per liter catalyst per hour.

The liquid product from the hydrocracking step was separated in a fractionator with an effective cut point (as determined by ASTM D2892-15 X2) of 319° C. into a light fraction comprising kerosene and a heavier fraction. The boiling point distribution of the light fraction comprising kerosene and heavier fraction was determined according to ASTM D-2887 and ASTM D-7169, respectively.

The heavier fraction was recycled to the hydrocracking step at a combined feed ratio of 1.47.

The weighted average bed temperature was adjusted until there was no net accumulation of the heavier fraction, i.e. until the flow of the heavier fraction was 0.47 times the fresh feed flow.

The light fraction comprising kerosene was distilled separately in a kerosene fraction and a light fraction (predominantly comprising C5-C8 naphtha) at an effective cut point of 125° C. based on ASTM D2892-15 X2 using boiling point distribution determined according to ASTM D-2887. The yield of the kerosene fraction was 70.9 wt. % based on the fresh C5+ fraction fed.

The properties of the kerosene fraction are shown in Table 1 below.

TABLE 1
Properties of kerosene fraction

| | Method | Spec¹ | Ex. 1 | Ex. 2 | Ex. 3 |
|---|---|---|---|---|---|
| Freezing | D-5972 | Max. −40 | −54.0 | −48.0 | −32.7 |
| point [° C.] | | | | | |
| Flash Point | IP170 | Min. +38 | 41.5 | 43.5 | 45.0 |
| [° C.] | | | | | |
| Boiling | D-86-18 | | | | |
| range | | | | | |
| IBP [° C.] | | | 150 | 151 | 152 |
| T10 [° C.] | | Max. 205 | 168 | 173 | 174 |
| T50 [° C.] | | Report² | 218 | 236 | 237 |
| T90 [° C.] | | Report² | 276 | 288 | 289 |
| FBP [° C.] | | Max. 300 | 291 | 302 | 302 |
| T90 − T10 | | Min. 22 | 108 | 115 | 115 |
| [° C.] | | | | | |
| SimDist | D-2887 | | | | |
| T10 [° C.] | | Report² | 144 | 151 | 152 |
| FBP [° C.] | | Report² | 322 | 332 | 333 |
| C16+ [wt. %] | GCxGC | | 24.0 | 38.5 | 35.8 |

¹Spec = specification according to ASTM D7566-20
²Report = the value needs to be reported on the Certificate of Analysis/Certificate of Quality of a fuel, but is not a minimum or maximum specification.

### Discussion

As can be seen from Table 1 above, by using a catalyst comprising a molecular sieve (with a pore size between 5 and 7 angstrom and a SiO₂/AlO₃ ratio of at least 25) and a group VIII metal in the hydroconversion step of a Fischer-Tropsch derived C5+ fraction, and by recycling the ‘wide-cut’ 319° C.+ fraction, a high yield (of above 70 wt. %) of a premium quality kerosene is obtained, without the need for additional catalytic dewaxing or additional oligomerization.

In this respect it is noted that, despite the presence of significant amounts of material boiling above 260° C. in the kerosene fraction, the freezing point is well below the minimum specification of −40° C. and even below the −47° C. specified for Jet A1.

The Final Boiling Point (FBP) of the kerosene of 291° C. (which is well below the maximum of 300° C. specified) indicates that further optimization of the kerosene yield is possible by optimizing the effective cut point between kerosene and recycled heavier fraction.

Further, the flash point of +44° C., which is well above the minimum of +38° C. specified, indicates that further optimization of the kerosene yield is possible by optimizing the cut point between kerosene and light fraction.

### Example 2 (Invention)

Similar to Example 1, a C5+Fischer-Tropsch product comprising 4.7 wt. % boiling below 150° C. and 67 wt. % boiling above 370° C. was continuously fed to a hydrocracking step at 60 bar g and a temperature of 343.4° C.

In the hydrocracking step the fresh C5+ fraction was supplied to a reactor comprising a stacked bed of a first catalyst below a second catalyst. The volume ratio of the second catalyst to the first catalyst was 4:1. The C5+ fraction contacted the second catalyst before contacting the first catalyst. The weight hourly space velocity (based on the total volume of catalyst in the reactor) was 1.0 kg fresh feed per liter catalyst per hour.

The first catalyst was the silica-bound, ammonium hexafluorosilicate-treated Pt/ZSM-12 catalyst from Example 1. The second catalyst comprised 0.8 wt. % platinum on an amorphous silica-alumina carrier.

The liquid product from the hydrocracking step was separated in a fractionator with an effective cut point (as determined by ASTM D2892-15 X2) of 328.5° C. into a light fraction comprising kerosene and a heavier fraction. The boiling point distribution of the light fraction comprising kerosene and the heavier fraction was determined according to ASTM D-2887 and ASTM D-7169, respectively.

The heavier fraction was recycled to the hydrocracking step at a combined feed ratio of 1.57. The weighted average bed temperature was adjusted until there was no net accumulation of the heavier fraction, i.e. until the flow of the heavier fraction was 0.57 times the fresh feed flow.

The kerosene fraction was distilled separately in a kerosene fraction and a light fraction at an effective cut point of 125° C. based on ASTM D2892-15 X2 using boiling point distributions determined according to ASTM D2887.

The yield of the kerosene fraction was 77.2 wt. % based on the fresh C5+ fraction fed.

The properties of the kerosene fraction are shown in Table 1 above.

### Discussion

As can be seen from Table 1 above, the use of the stacked bed and by recycling the ‘wide-cut’ 328.5° C.+ fraction allows for the production of a high yield (of above 70 wt. %) of a premium quality kerosene with freezing point below −47° C. (meeting the Jet A1 standard). This, without the need for additional catalytic dewaxing or additional oligomerization.

In this respect it is noted that, despite the presence of significant amounts of material boiling above 260° C. in the kerosene fraction, the freezing point is well below the minimum specification of −40° C. and even below the −47° C. specified for Jet A1.

The D86 Final Boiling Point (FBP) of the kerosene of 302° C. for Example 2 is just above the maximum of 300° C. specified. The person skilled in the art will readily understand that further optimization of effective cut point and/or the separation sharpness between kerosene and recycled heavier fraction will be possible to result in a FBP below the maximum of 300° C. specified. Such optimization will have only a small impact on the obtained yields, while a reduction of FBP is not expected to result in worsening of freezing point.

Further, the flash point of +43.5° C., which is well above the minimum of +38° C. specified, indicates that further optimization of the kerosene yield is possible by optimizing the cut point between kerosene and light fraction.

### Example 3 (Comparative)

The experiment of Example 2 was repeated, except that:
the reactor in the hydrocracking step comprised only the second catalyst comprising 0.8 wt. % platinum on an amorphous silica-alumina carrier as described in Example 2;
the temperature in the hydrocracking step was 344.1° C.; and
the effective cut point between the light fraction comprising kerosene and the heavier fraction was 329° C.

The yield of the kerosene fraction was 77.2 wt. % based on the fresh C5+ fraction fed.

The properties of the kerosene fraction are shown in Table 1 above.

### Discussion

The data for comparative Example 3 in Table 1 above show that, using only the second catalyst, it is not possible to obtain a kerosene fraction with a high amount of C16+ that meets the freezing point specification of maximum −40° C., let alone −47° C.

To meet the freezing point specification of maximum −40° C., the kerosene fraction would need to be cut lighter (possibly by taking the 140-260° C. cut), but this would go at the expense of the kerosene yield and move this to well under 70 wt. %. It is estimated that the kerosene yield, when using only the second catalyst, would then be in the range of 50-60 wt. %, based on the fresh C5+ feed.

The person skilled in the art will readily understand that many modifications may be made without departing from the scope of the invention.

1. A process to prepare kerosene, the process at least comprising the steps of:
(a) providing a syngas stream comprising hydrogen (H₂) and carbon monoxide (CO);
(b) subjecting the syngas stream provided in step (a) to a Fischer-Tropsch reaction thereby obtaining a Fischer-Tropsch product comprising at least 50 wt. % of compounds boiling above 370° C.;
(c) separating the Fischer-Tropsch product into at least a C1-C4 fraction, H₂O and a C5+ fraction;
(d) subjecting the C5+ fraction as separated in step (c) to hydroprocessing thereby obtaining a mixture comprising at least a kerosene fraction and a heavier fraction;
(e) separating the mixture as obtained in step (d) thereby at least obtaining the kerosene fraction and the heavier fraction, wherein the kerosene fraction has a Final Boiling Point of at most 302° C., preferably at most 300° C. as determined by ASTM D86-18 and a flashpoint of at least 38° C. as determined by IP170,
wherein in the separation of step (e) the effective cut point, as determined by ASTM D2892-15 X2, between the kerosene fraction and the heavier fraction is at least 315° C. and at most 330° C., and
wherein in step (e) a kerosene yield of above 70 wt. % is obtained, based on the weight of the C5+ fraction subjected to hydroprocessing in step (d);
(f) recycling at least a part of the heavier fraction as obtained in step (e) to the hydroprocessing of step (d);
wherein step d) is carried out by contacting the C5+ fraction with a first catalyst, wherein the first catalyst comprises a molecular sieve with a pore size between 5 and 7 angstrom and a SiO₂/Al₂O₃ ratio of at least 25 and a group VIII metal.

2. A process according to claim 1, wherein at least a part of the C1-C4 fraction obtained in step (c) is converted into syngas and combined with the syngas stream provided in step (a).

3. A process according to claim 1, wherein the hydroprocessing in step (d) takes place at a pressure of above 20 bara.

4. A process according to claim 1, wherein the first catalyst comprises a molecular sieve, and platinum or palladium as Group VIII metal and wherein the molecular sieve is a MTW, MTT, TON type molecular sieve or ZSM-48 or EU-2.

5. A process according to claim 1, wherein in step (d) the C5+ fraction is contacted with a second catalyst before contacting with the first catalyst, wherein the second catalyst comprises a Group VIII noble metal supported on an amorphous acidic carrier.

6. A process according to claim 1, wherein in step (d) the C5+ fraction is contacted with a second catalyst after contacting with the first catalyst, wherein the second catalyst comprises a Group VIII noble metal supported on an amorphous acidic carrier.

7. A process according to claim 5, wherein the amount of catalyst as used in step d) comprises 10-90 vol. % of the first catalyst and 90-10 vol. % of the second catalyst.

8. A process according to claim 1, wherein the kerosene fraction obtained in step (e) has a freezing point of at most −40° C. as determined by ASTM D5972.

9. A process according to claim 1, wherein the kerosene fraction obtained in step (e) has an amount of C16+ of at least 5 wt. %.

10. A process according to claim 1, wherein in the separation of step (e) the effective cut point, as determined by ASTM D2892-15 X2, between the kerosene fraction and the heavier fraction is at most 325° C.

11. A process according to claim 1, wherein in step (e) also a second C1-C4 fraction and a naphtha fraction are obtained, wherein at least a part of the second C1-C4 fraction and/or the naphtha fraction are converted into syngas and combined with the syngas stream provided in step (a).

12. A process according to claim 1, wherein in step (e) the separation is done by atmospheric distillation.

13. A process according to claim 1, wherein the kerosene fraction is obtained without a separate catalytic dewaxing or oligomerization step, and wherein the hydroprocessing in step d) takes place in a heavy paraffinic conversion unit, preferably without subsequent separation of normal and iso-paraffins.
